(19) United States
(12) Patent Application Publication
(10) Pub. No.: US 2009/0209896 A1
(43) Pub. Date: Aug. 20, 2009

(54) Method and apparatus for time-dependent and temperature-dependent clinical alert

(76) Inventor: James R. Selevan, Laguna Beach, CA (US)

Correspondence Address: JAMES R. SELEVAN, 1661 SUNNSET RIDGE DRIVE, LAGUNA BEACH, CA, 92651, US

(21) Appl. No.: 12/322,010

(22) Filed: Jan. 28, 2009

Related U.S. Application Data
(60) Provisional application No. 61/066,427, filed on Feb. 19, 2008.

(51) Int. Cl. A61F 15/00; A61B 5/01; G04F 10/00

(52) U.S. Cl. 602/41; 600/549; 368/109

(57) ABSTRACT

Devices and methods are disclosed for measuring a pre-defined period as a method to alert medical personnel of the need for clinical intervention. Many invasive and non-invasive medical procedures require constant monitoring or intervention. Failure to properly observe or intervene may result in clinical harm. The devices and methods disclosed herein are useful in any field of medicine where periodic monitoring or procedures are required. The devices is designed to be familiar to medical personnel, and in its most common form will be packaged as a band aid, but may also be packaged as a coin sized device that adheres to or clips onto a patient's bandage, limb, or on a necklace. The timer provides an audible warning of timeout, a visual indication of timeout, a tactile indication or any combination of the three. The number of hours in the countdown sequence is variable and determined from a series of choices provided to the caregiver. The device can be used to monitor temperature and warn of an infection.

### PRIORITY CLAIM

This application claims priority benefit under 35 USC §119(e) from U.S. Provisional Application No. 61/066,427, filed Feb. 19, 2008, entitled METHOD AND APPARATUS FOR TIME-DEPENDENT AND TEMPERATURE-DEPENDENT CLINICAL ALERT, the entire contents of which is hereby incorporated herein by reference.

### FIELD OF THE INVENTION

The field of this invention is general medicine, family practice, pediatric medicine, and cardiology, Surgery, intensive care, emergency medicine and departments, operating rooms, medical surgical wards, pediatric intensive care, radiology, surgery, clinical laboratory, microbiology laboratory, and all aspects of medical and veterinary patient/animal care. More specifically, the field relates to devices and methods for notifying clinical personnel of time-dependent and temperature-dependent protocols, procedures, and necessary clinical patient or, in the case of veterinary medicine, animal intervention.

### BACKGROUND OF THE INVENTION

Many procedures and interventions are performed on human and animal patients that require monitoring and reassessment on a periodic basis. Examples include wound checks, intravenous line replacement, central catheter line replacement, arterial line placement, skin examination for advancement of an infected margin, joint examination, neurologic examination, vital signs, administration of intravenous, oral, or rectal medications, central nervous system pressure monitoring or catheter replacement, etc. Other areas of interest include the clinical chemistry laboratory and microbiology laboratory, where specimens and cultures often are staged for analysis while awaiting completion of growth or a chemical reaction. Postoperative protocols vary by surgeon, and medical/surgical nurses must be made aware of the individual preferences dictated by each surgeon with regard to the timing of wound change or inspection, range of motion activity, etc. Maintaining schedules of these important procedures is the responsibility of the physician, laboratory technician, and nurse caring for the patient or animal. Often the recognition of the need to change a bandage, examine a wound, or to reinsert an intravenous line, examine a culture, as examples, goes unnoticed. This protocol variation results from the busy nature of the hospital, laboratory, or clinical setting, and lack of organizational processes and oversight to keep track of these time-critical procedures. The consequences of such oversight and failure to change an intravenous line, as one example, can be severe.

The risk of infection from an intravenous line is well documented in the medical literature, and the likelihood of infection grows with the length of time that the intravenous line is in place. As a result, the community standard of care defines an acceptable period for an intravenous line placement, or a wound dressing, or a central venous line placement, or an arterial line placement, etc. Typical acceptable periods include 12, 18, 24, 36, 48, 72 hours, 96 hours, etc. At the end of this period, the intravenous line must be removed and reinserted in a different location in order to minimize the risk of infection. The same is true for examining wounds, replacing arterial lines, central venous catheter lines, central nervous system lines, etc. Laboratory procedures often require 30 minute or 1 hour delays while blood is allowed to clot, or chemical reactions are allowed to come to completion. It is clear and well documented in the medical literature, that failure to carefully monitor the time duration of all of these medical/veterinary procedures results in more infections, delay in laboratory reporting, and increased cost of care. Hence, any method or device that helps to insure proper time-management and appropriate clinical intervention will be sought after and well received by the medical community. Patient care could be greatly improved if an improved method of keeping track of wound care, laboratory procedures, or catheter placement time could be developed.

### SUMMARY OF THE INVENTIONS

These inventions relate to improved devices and methods for 1) the organization of scheduling and time-management of important medical procedures and examinations, and 2) determination of skin temperature as a trigger to alert medical personnel. The devices and methods aid medical personnel in performing the necessary task at the appropriate time. The devices, more generally, act as a reminder for both medical personnel and for the patient (in the case of humans) to perform a necessary and important procedure, and to alert appropriate personnel that the skin temperature is elevated. It is also important that such methods or devices be un-encumbering, easy to use, and low cost. The methods or devices can also aid the patient directly, but reminding the patient of the need to either intervene directly, as in changing a bandage, or to remind medical personnel of the need to intervene. In this way the patient enters the process as the “final line of defense” against medical errors. In addition, when used in the clinical or microbiology laboratory, the device will allow laboratory personnel to identify time-critical events, thereby reducing the time and cost associated with the process. Young children and adolescents that use ambulatory insulin pumps often forget to refill the reservoir. Application of this device to these insulin pumps, or on the skin next to the pump, would act as a reminder to refill with insulin.

The inventions comprise a timing device, battery, and transducer, packaged in a familiar medical device such as a Band-Aid (proprietary name), band-aid, wrap, clip, or packaged as an approximately “dime-sized”, or “quarter-sized”, button that adheres to a bandage, blood vial, Petri dish, etc. The devices are designed to measure a predetermined interval, and when this interval has elapsed, to alert the medical personnel or patient (in the case of a human) to perform a procedure. In addition, the device measures temperature on the skin surface, and alerts appropriate personnel should the temperature rise or fall below a predetermined threshold. Furthermore, a key element of the design is calculation of the area under the curve of temperature versus time. Estimating core body temperature by measuring skin temperature is known to be inaccurate. Hence, a change in skin temperature, up or down, rather than estimate of core temperature, may provide the clinician with useful information in the absence of core temperature. There are critical physiologic events that will either raise or lower skin temperature. However, minor variations of skin temperature may occur owing to change in room temperature, patient movement, etc. Establishing a threshold or “delta” alone may introduce false positive alarms. However, when measured over time, that is, as the product of temperature and time, then a true change in skin temperature may be more accurately established and more predictably represent a physiologic event. The output transducer that actually alerts personnel, that is, the method of converting electrical information to sensory information, can comprise one or more Light Emitting Diodes (LED), sound generating devices (beeper), vibration devices, etc. At the end of the predetermined interval, or should the skin temperature rise or fall greater than a certain defined delta, change, or threshold, the band aid or button will light up, beep, or vibrate, hence sending an alert to the appropriate personnel that a procedure is required, and specifically, required at the anatomical location of the placement of the device. In other embodiments, should the product of the time and skin temperature rise or fall greater than a defined change interval or threshold, the band air or button will trigger an alert or signal output. Different timing intervals can be denoted by different colors of packaging, or may be programmed by the medical personnel at the time of activation. For example, once activated, 1 press of a small button could designate a 1-day timing interval, while 2 presses could signify 2-days, 3 presses could represent 3-days, etc. In addition, the bandage or clip can comprise a unique color, designating the period timed. Different temperature levels can be denoted by different colored LEDs or different flashing sequence of the LEDs.

For example, at insertion of an intravenous line in a patient's arm, the medical technician, nurse, physician, etc., would place this device (in the form of a band aid, as an example) on top of the bandaged intravenous entry site on the arm. The device can be made available in many different timing intervals. If the medical personnel prefers that the intravenous line be removed and relocated in 24 hours, for example, then the technician would have used a 24-hour band-aid alert device. At the conclusion of the 24-hour period, the band-aid will begin to flash with activation of one or more LEDs, indicating to medical personnel that the intravenous line must be removed and relocated. The LEDs can continue to flash for up to about 10 days, or more, if not removed and discarded. This alert is evident to all personnel that come into contact with the patient, as well as the patient and their family. The frequency of illumination of the LEDs, that is, the rate at which they flash, is designed to provide additional information. For example, the LED could flash at a 1-per-second rate during the first 15 minutes after it is triggered. At 15 minutes, the rate could decrease to 1-per-2 seconds, then 1-per-3 seconds, etc. An alternative would be a repeating series of flashes, such as 1, for the first 15 minutes, 2 for the second 15 minutes, 3 for the third 15 minutes, etc. If the wound becomes infected, or if the patient develops a fever, then a red (for example) LED would begin flashing, warning all personnel that the patient has a clinical problem. The sequence of the flash of the LED could denote the degree of temperature elevation. For example, a single flash every 5 seconds could denote a 1 degree elevation of temperature. A series of 2 flashes every 5 seconds would denote a 2 degree elevation in temperature, etc.

The invention comprises a battery, timing circuitry, micro-controller, temperature transducer, one or more LEDs or other visual output devices, a beeper, a vibration device, or other warning system. It can be packaged in the form most convenient for use by the medical personnel. In an embodiment, the device configuration is that of a band-aid. In another embodiment, the device is configured as a small, dime-sized, or quarter-sized, device that mechanically clips to a patient's clothing, adheres to a bandage with an adhesive, or hangs on a limb or around the neck of a patient. The device can be disposable or reusable. The device can be sterilized for bacterial and viral-free environments, such as the operating room, emergency room, procedure room, radiology suite, delivery suite, etc. using standard sterilization methodologies such as, but not limited to, steam sterilization, gamma irradiation, electron beam irradiation, ethylene oxide, and the like.

In addition, the device can encompass two (2) or more LEDs or other visual displays, such that various time intervals may be monitored, and the procession of time can be tracked. For example, a green, yellow, and red LED could be used in the band-aid, such that at the end of 12 hours the green LED illuminates, at the end of 24 hours the yellow LED illuminates, and at the end of 36 hours the red LED is lit up. This method provides additional information and timing for the clinical personnel including the amount of time passed since the band-aid/bandage should have been changed. At the end of the time period the band-aid could beep to provide additional alerts to personnel. Should the temperature exceed a threshold considered abnormal, a red (or other color) LED could flash in a different temporal pattern that indicates the degree of temperature elevation.

The timing sequence can be initiated by removing the adhesive-protection material (as in “peeling off the back of the band aid) or engaging some other type of electrical switch. As the protective sheet is removed from the band-aid, the battery contacts are exposed to the circuitry, which initiates the start-up alert sequence. An alternative would be for a momentary contact switch to “wake up” the microcontroller from a “sleep mode”. In this embodiment, the battery can be permanently connected to the circuit at the time of manufacture. In an embodiment, the band-aid or button's visual indicator rapidly flashes for about 20 seconds, thereby informing the medical personnel that the device is armed and working. In addition, the sequence of the start-up flash can inform the user of the preprogrammed time period. The band-aid or clip is then attached to the patient's dressing. After the 20-second start-up flash sequence, the band-aid or button stops flashing or beeping. At the end of the predetermined timing period, for example 24 hours, or if the patient's temperature should exceed a threshold, the band-aid will again flash, beep or vibrate, thereby alerting the medical personnel or patient that intervention is necessary.

In addition, should a wound become infected, there are four clinical signs that must be recognized by the clinician. These are 1) erythema (redness), 2) swelling, 3) warmth, and 4) pain. Hence, automated recognition of the presence of an infection that is obscured by a bandage is quite useful. Modern technology, in the form of appropriate transducers and logic, enable the design of a device that will detect subtle elevations of temperature in a localized area. Such a device could alert the clinician of an infection early in its course, thereby preventing deterioration and sepsis. In an embodiment, the device comprises a thermal sensor; a threshold monitor; micro-controller; a power supply; interconnect electrical lines; and audio or visual display components to notify a caregiver that an active infection is in progress.

Body temperature elevated above the norm is considered a sign of inflammation or infection. The traditional tool for measuring temperature, either the mercury or electronic thermometer, requires interaction and participation of the patient. These modern devices sense external ear canal temperature, rectal or oral temperature. In all cases, assessment of body temperature requires disturbing the patient, and active intervention by medical personnel. Continuous monitoring of body temperature currently uses a transducer attached to the body, and wires running to a control unit that displays the temperature. The device envisioned in this application allows body temperature to be continuously monitored without the need of wires attached to the patient.

For purposes of summarizing the inventions, certain aspects, advantages and novel features of the inventions are described herein. It is to be understood that not necessarily all such advantages may be achieved in accordance with any particular embodiment of the inventions. Thus, for example, those skilled in the art will recognize that the invention may be embodied or carried out in a manner that achieves one advantage or group of advantages as taught herein without necessarily achieving other advantages as may be taught or suggested herein.

These and other objects and advantages of the present inventions will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1A illustrates a bottom view of a bandage having a central gauze area, an adhesive region, a timing and signaling device, and gas vents, according to an embodiment of the invention;

FIG. 1B illustrates a bottom view of a bandage having a central gauze area, an adhesive region, a timing and signaling device, and an adhesive cover strip, according to an embodiment of the invention;

FIG. 1C illustrates a top view of the bandage of FIGS. 1A and 1B wherein the bandage includes a timing and signaling device affixed to its top surface, according to an embodiment of the invention;

FIG. 2A illustrates a top view of a circuit board, timer, temperature sensor, battery compartment, and LEDs integrated into a module suitable for being affixed to a bandage, according to an embodiment of the invention;

FIG. 2B illustrates a top view of a bandage comprising an adhesive region, a gauze pad, an extension of the adhesive cover strip, and the module of FIG. 2A, according to an embodiment of the invention;

FIG. 3A illustrates a top view of a timing and display module integrated to a band-aid type bandage, wherein the module further includes a temperature sensor, battery connections, LEDs and battery isolation mechanism, according to an embodiment of the invention;

FIG. 3B illustrates a side cross-sectional view of the timing and display module integrated into the bandage of FIG. 3A along section A-A to show the thickness profile and the interaction of the cover strip remnant with the battery isolation mechanism, according to an embodiment of the invention;

FIG. 4A illustrates a top view of a small, flat, button-like, cylindrical timing module with adhesive backing for use with laboratory test apparatus, according to an embodiment of the invention;

FIG. 4B illustrates a side view of the small, flat cylindrical timing module of FIG. 4A, according to an embodiment of the invention;

FIG. 5A illustrates an oblique view of the small, flat cylindrical timing module of FIG. 4A having a label suitable for specifications or advertising and an adhesive attachment means, according to an embodiment of the invention;

FIG. 5B illustrates an oblique view of the small, flat cylindrical timing module of FIG. 5A but replaces the adhesive region with a clip suitable for attachment to the clothing of a patient or bed linens, according to an embodiment of the invention;

FIG. 6A illustrates a view of the timing module attached to a cord or lanyard, suitable for being worn around the neck like a necklace, according to an embodiment of the invention;

FIG. 6B illustrates a view of the adhesive backed timing module of FIG. 5A stuck to the cover of a Petri dish and suitable for timing completion of a grow-out cycle in the Petri dish, according to an embodiment of the invention; and

FIG. 7 illustrates a plastic snap-on device, or cover, according to an embodiment of the invention.

### DETAILED DESCRIPTION OF THE INVENTIONS

In accordance with one or more embodiments of the present invention, a clinical timing device, packaged in the form of a band-aid or clip, and accessory components are described herein. In order to fully specify this preferred design, various embodiment specific details are set forth, such as the number and makeup of the timing intervals, activation mechanisms, and the like. It should be understood, however that these details are provided only to illustrate the presented embodiments, and are not intended to limit the scope of the present invention.

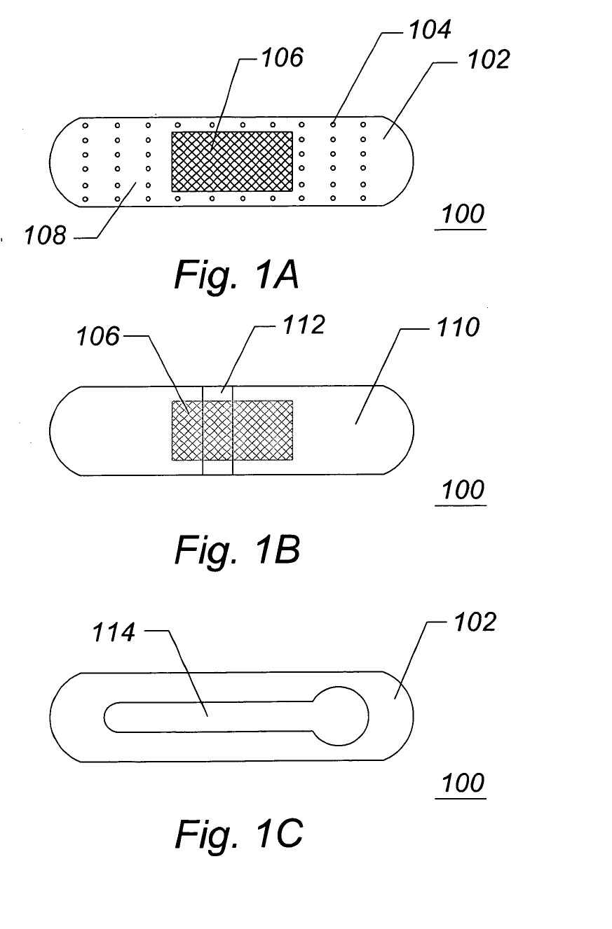

FIG. 1A illustrates a bottom view of an electronic band-aid or timing bandage 100 and the circuitry of an embodiment of the invention to be adhered to the bandage. The bottom of the timing bandage 100 comprises a backing or substrate 102, a plurality of breathing holes 104, a central gauze pad 106, and an adhesive layer 108.

Referring to FIG. 1A, the gauze pad 106 is affixed to the center of the timing bandage backing 102 by bonding, welding, adhesive, or the like. The adhesive layer 108 is adhered to the backing 102. The plurality of breathing holes 104 can beneficially be integral to the backing 102 and pass completely through the backing 102.

FIG. 1B illustrates the timing bandage or band-aid 100 of FIG. 1A further comprising the gauze pad 106, an adhesive cover strip 110, and a cover strip fold 112 to allow easy removal of the cover strip 110.

Referring to FIG. 1B, the cover strip 110 is a non-wettable polymeric material that lightly sticks to the adhesive layer 108 but can be removed by grasping the cover strip fold 112 and pulling the cover strip off of the adhesive layer 108. The cover strip 110 is suitable for packaging and storage of the bandage 100 to preserve the adhesive qualities of the adhesive layer 108 and to prevent contamination from reaching the adhesive layer 108.

FIG. 1C illustrates a top view of the bandage 100 further comprising the backing 102 and the timing module 114.

Referring to FIG. 1C, the timing module 114 comprises a flexible printed circuit board and components that is affixed to, or embedded within, the band-aid substrate 102. Thus, the timing bandage 100 retains its flexibility but now gains the ability to actively monitor time, temperature, or other parameters and signal the caregiver of an end of time, out of range temperature, out of range time temperature product, out of range time temperature integral, and the like.

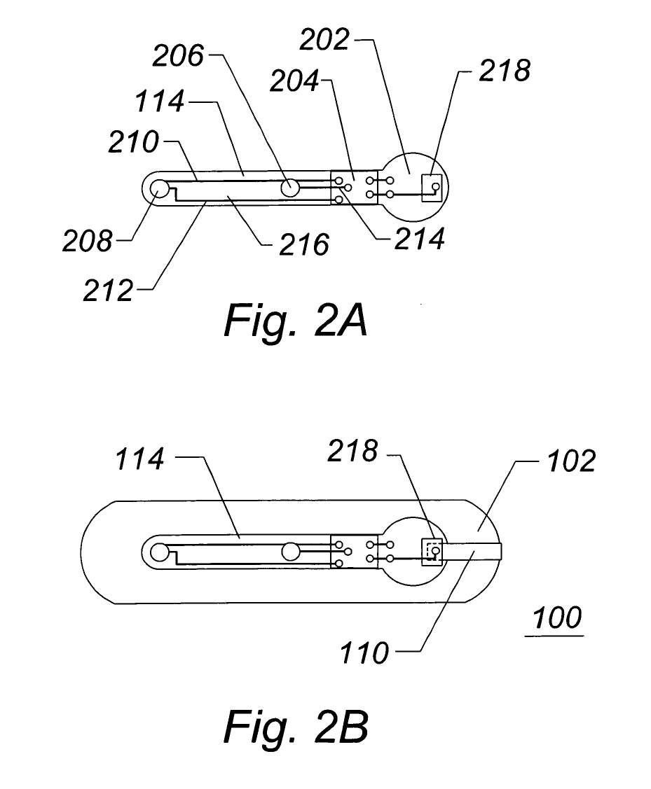

FIG. 2A illustrates details of the circuit board 114. The circuit board 114 comprises a battery interrupt device 218, a battery housing 202, a controller 204, a first LED 206, a second LED 208, a circuit substrate 216, a common electrical bus 210, a first LED power bus 214, and a second LED power bus 212.

Referring to FIG. 2A, one or more battery-interrupt devices 218 insulate, or electrically isolate, the battery (not shown) during storage. Referring to FIG. 1B, upon removing the adhesive protection material 110 (“peeling the band aid”), a remnant of the adhesive protection material 110 is removed from the battery interrupt device 218 causing the battery (not shown) to come into spring-loaded electrical contact with the circuitry to activate the device 114. Activation is denoted by a short sequence of flashes of the LEDs 206 and/or 208.

FIG. 2B illustrates a top view of the timing bandage 100 comprising the circuit board 114 affixed to a bandage substrate 102. The remnant of the adhesive protection material 110 is illustrated in place electrically isolating contacts within the battery interrupt device 218. The circuit board 114 can be flexible and can be affixed to, glued to, welded to, bonded to, embedded within, or otherwise attached to the flexible bandage substrate 102.

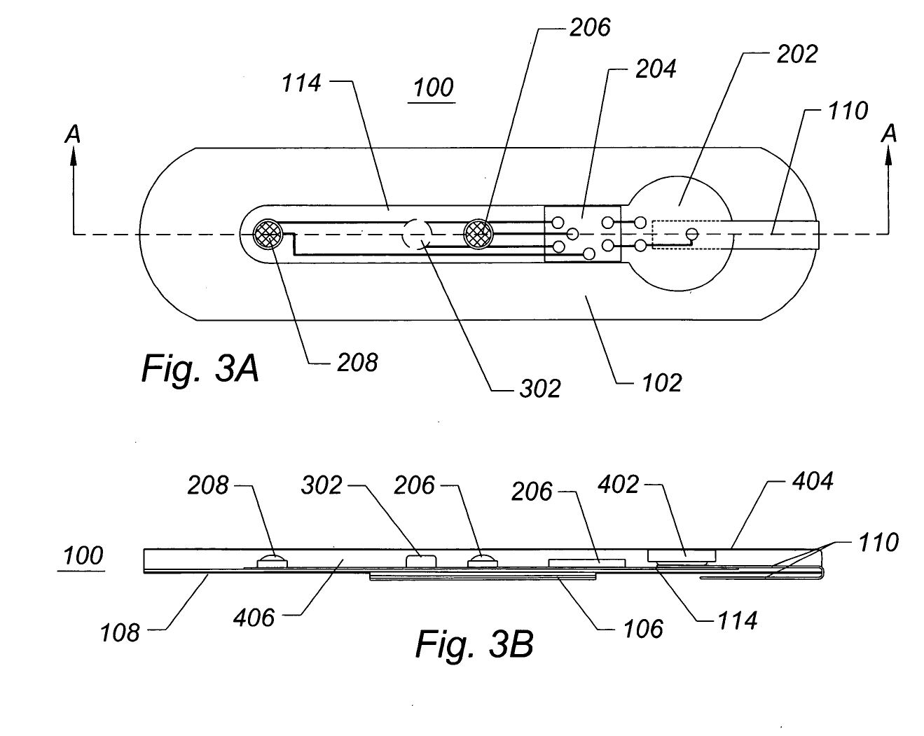

FIG. 3A illustrates a top view of the timing bandage 100 comprising the substrate 102, the circuit board 114, the controller 204, the battery compartment 202, the first LED 206, the second LED 208, and a thermal sensor 302.

Referring to FIG. 3A, the control circuitry 204 is located on the component board 114, and connected to the first and second LEDs 206 and 208, respectively, along with the temperature sensor 302, via appropriate conductors. The control circuitry 204 comprises a timing mechanism, a power supply regulator, output power switching, temperature sensing and monitoring, threshold detection circuitry, user input handling, and the like. The temperature sensor 302 can comprise a thermistor, a thermocouple, infrared sensor, or the like. The control circuitry 204 can further comprise a microprocessor or micro-controller, memory, and software or firmware. The control circuitry 204 can be configured to display warning information upon reaching appropriate pre-set, or set-at the time of use, time intervals, time temperature products, time temperature integrals, or temperature levels that either exceed either norms or initially measured patient readings by a fixed amount or by a percentage of the initial or normal readings, or fall below a threshold as when the skin cools.

Referring to FIG. 3A, the electrical circuit card 114 can comprise a power supply 202, an on-off switch (not shown), an optional audio output device (not illustrated), a clock (not shown), a logic circuit (not shown), an optional visual output device such as a backlit, alphanumeric LCD display (not shown), one or more countdown start switches (not shown), and a rate input device (not shown), all of which are electrically interconnected by an electrical bus (not shown) on the circuit card 114.

The electrical subsystem of the timer bandage 100 is housed within the cover 404 (refer to FIG. 3B) of the timer bandage 100. The electrical subsystem comprises the aforementioned components, such as the power supply, a relay, the clock, and the logic circuit 206, and preferably comprises a chassis or circuit card 114 to which all components are mechanically connected. The components are electrically interconnected by the electrical bus comprised by the circuit card 114. The power supply 202 can preferably be a battery. The power supply 202 is preferably removably affixed to the electrical subsystem, but may be advantageously non-removable in other embodiments. The power supply 202 supplies power to the electrical bus of the electrical subsystem and is preferably switched by a sheet 110 that insulates the battery 202 from the circuit board 114 or a sheet 110 that separates contacts within a switching system 218 (FIGS. 2A and 2B). In another embodiment, the battery can be permanently, operably connected to the circuit such that the electrical device is always “powered-up”. In this design a simple momentary contact can be generated to represent a signal (interrupt) to the microcontroller instructing it to wake up and begin the timing sequence and notification protocols.

FIG. 3B illustrates a side view of the timing bandage 100 taken in cross-section A-A as illustrated in FIG. 3A. The timing bandage 100 comprises the adhesive cover strip 110, a cover seal 404, a battery 402, the timing circuit 206, the gauze pad 106, the circuit board 114, the first LED 206, the second LED 208, the temperature sensor or measuring device 302, the adhesive layer 108, and the timing system embedment 406.

Referring to FIG. 3B, the adhesive cover strip 110 is illustrated partially covering the adhesive layer 108 while the remnant of the cover strip 110 is illustrated separating the battery 402 from a contact on the circuit board 114. The timing bandage 100 can also comprise an audio output device (not shown) such as, but not limited to, a small loudspeaker, a buzzer, a bell, or other audio signaling device. The audio output device can be programmed to beep, ring, buzz, or generate simple or complex tones at varying rates and can even be programmed to play voice recognizable signals such as but not limited to “12 HOURS ELAPSED”, “THE PATIENT HAS A FEVER OF 102 DEGREES”, “THE BATTERY IS LOW”, or the like. The timing system embedment 406 can be flexible foam, preferably open cell, it can comprise a gel, or it can be an elastomeric or otherwise deformable, soft pad. The timing system embedment 406 is preferably porous to gas penetration so that the skin underneath can breathe. The circuit board 114 comprises the bus wires 210, 214, 212, etc., illustrated in FIG. 2A, which operably interconnect the electrical terminals of the various circuit board 114 components.

The printed circuit board 114 can be either rigid or flexible, depending on the final packaging. In the case of the band-aid 100, the printed circuit board 114 can be flexible. The countdown sequence start mechanism is a sheet of material 110 that separates the battery contact from the circuitry 218. Referring to FIGS. 1A and 1B, upon peeling off the adhesive cover sheet 110 protecting the adhesive 108, the battery 402 is allowed to contact wiring on the printed circuit board 114. The visual, auditory, or tactile output devices 206 and 208 are attached to the printed circuit board 114. Advertising information and timing period can be printed on the non-skin side of the band-aid or clip 110. The power supply 202 and the timer circuit 204 are affixed to the printed circuit board 114. The power supply 202 preferably comprises a battery 402, which may be removable or replaceable, or may be sealed to preclude replacement.

The packaging material for the timer bandage 100 will be such that it does not cause a dermatologic reaction. The choice of material will be defined by conventional manufacturing techniques and experience. The device and its packaging may be sterilized for to allow use in those environments where sterility is critical, such as operating rooms, obstetric delivery suites, radiology and cardiology suites, etc. Sterilization can be performed using gamma irradiation, steam sterilization, ethylene oxide, and the like.

Referring to FIGS. 1A, 3A, and 3B the materials used in the manufacture of the timer circuit board 114 include but are not limited to polymers such as polyvinyl chloride, PEBAX, acrylonitrile butadiene styrene, PETG, PET copolymers, polyurethane, polyester, polyethylene, polypropylene, polytetrafluoroethylene, polyether etherketone (PEEK), fluorinated ethylene propylene, polytetrafluoroethylene-perfluoromethylvinylether, silicone rubber, and the like, as well as any other material used in the standard manufacturing of rigid or flexible printed circuit boards.

The countdown sequence start actuator 218, illustrated in FIG. 2B, may be related to battery isolation via a sheet of polymeric or fibrous material insulating the battery from the circuit board, or there can be a user initiated button (not shown) on the outside of the timing bandage 100 such that a push button, tap of the finger, toggle switch, or twist to engage the battery 402 might activate the device. The timer bandage 100 can be waterproof in certain embodiments, although in many environments and clinical applications this will not be necessary.

The visual output devices 206 and 208 are comprised of light emitting diodes in a preferred embodiment. The visual output devices may further comprise devices such as, but not limited to, LCD displays, active matrix displays, light bulbs, and the like. More than one visual output device 206 and 208 is advantageously used to communicate system status to the user. In the simplest embodiment, the visual output device 206 or 208 blinks when the countdown sequence has reached zero, or when the clock arrives at the pre-determined elapse time. In a more sophisticated embodiment, the visual output device 206 or 208 illuminates with one color when the sequence achieves one time period and with another color when the sequence reaches another milestone period. In yet another embodiment, the visual output device 206 or 208 flashes at different rates or with different colors when the temperature sensor detects a rise or fall in skin temperature. The flashing rates may vary from 0 Hz, or continuously on, to rates as high as 100 Hz. The visual output device 206 or 208 in yet another embodiment indicates the status of the battery and whether or not it requires replacement. In yet another embodiment, the visual output device 206 or 208 displays alphanumeric information relating to parameters such as, but not limited to, the date, time remaining, time last checked by medical personnel, the battery status, the length of the timing interval, and the like.

Referring to FIG. 3B, the power supply 202 preferably comprises a battery 402. The battery or set of batteries 402 are standard, easily replaceable cells such as those fabricated from chemistries such as, but not limited to, alkaline, lithium, nickel metal-hydride, lead acid, and the like. The batteries may be non-rechargeable or they may be rechargeable using a plug attached to the timer circuit card 114 or by placing the timer circuit card 114 near a charger that comprises a coil capable of inducing a field within the timer 114 that charges the battery 402. Small flat batteries such as those used in watches are appropriate as are batteries such as AAA batteries sold commercially. The power supply 202 is preferably able to provide voltages to the timer 114 ranging from 1.2 to 12 volts and more preferably between 1.2 and 6 volts.

The timer circuit 114 receives its power from the power supply 202 when the adhesive protective cover 110 is peeled from the timer band-aid 100. The timer circuit 204 may further receive inputs from wireless sources such as, but not limited to, those generated by microwave, radio waves, ultrasound, infrared, and the like. The timer circuit can be permanently, operably, electrically connected to a battery. In this embodiment, the microcontroller “wakes up” upon a momentary contact that sends a signal to the appropriate input pin of the microcontroller.

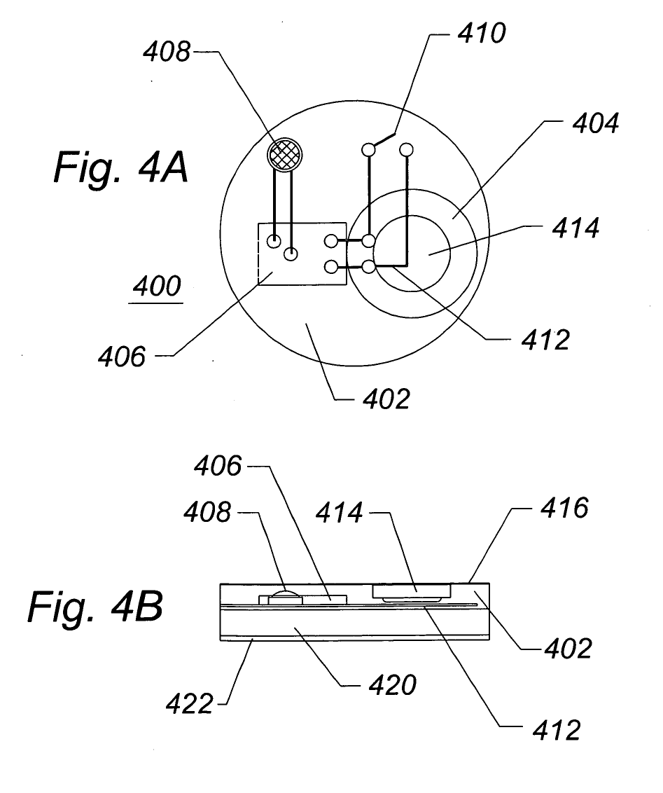

FIG. 4A illustrates another embodiment of a timing button 400, wherein the timing button 400 comprises an LED 408, a battery 414, a battery compartment 404, a switch 410, a controller 406, and electrical interconnects 412, all of which are affixed to a timer substrate 402.

Referring to FIG. 4A, the timing button 400 can function in the same way as the timer bandage 100 except that the button can be used in conjunction with various processes that require timing, not just wound dressing applications.

FIG. 4B illustrates a side view of the timing button 400 showing the battery 414, the timer substrate 402, the control circuit 406, the circuit board 412, an LED 408, a foam backing 420, and an adhesive layer 422. The timing button 400 can also comprise a cover shell 416, which can be transparent, translucent, or partially opaque. The cover shell 416 provides the function of water resistance or watertight seal. The timer button 400 can also comprise a cover strip (not shown) that covers the adhesive layer 422 during shipping and storage and which serves the same function as that of the cover strip 110 in FIGS. 3B, 1A, and 1B.

Referring to FIG. 4B, the foam backing 420 is preferably fabricated from conformable, soft foam, with a low spring-back force so that the foam takes the shape of any irregular object to which it is affixed by the adhesive layer 422. The adhesive layer 422 can preferably be aggressive but maintain high internal coherence so that when the timer button 400 is removed from an object, no, or little, adhesive residue remains. The foam backing 420 can comprise polyurethane foam, polycarbonate foam, or the like. The timer button 400 can also comprise an audio output device (not shown).

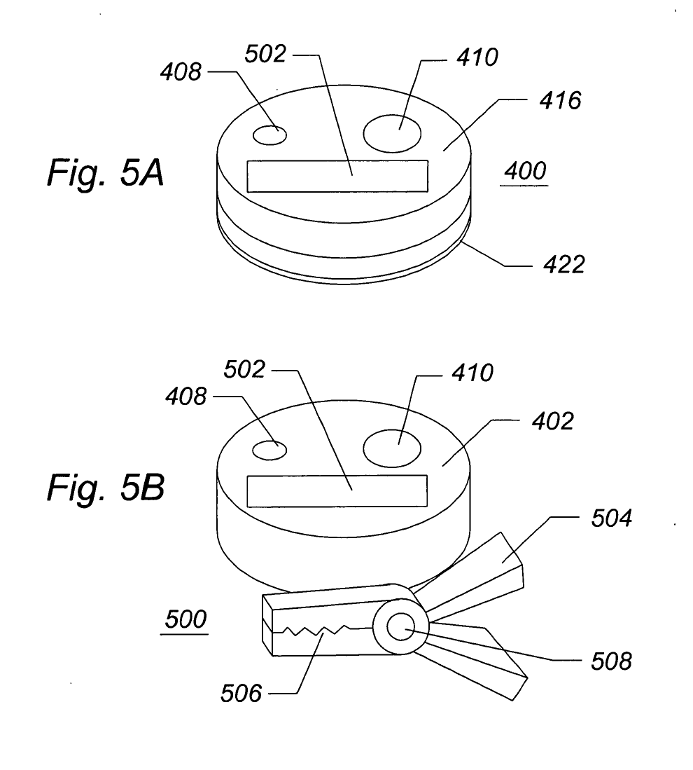

FIG. 5A illustrates an oblique view of the timer button 400 of FIGS. 4A and 4B. The timer button 400 comprises the cover shell 416, an on-off switch 410, an LED 408, a label 502, and the adhesive layer 422.

Referring to FIG. 5A, the advertising logo or label 502 is a primary feature of the protective layer or cover shell 416. The advertising logo 502 is a label affixed to the cover shell 416. The advertising logo 502 can be pad printed, printed, lithographed, holographically printed, Laser printed or etched, embossed, molded in with raised letters, and the like. The advertising logo 502 can be fabricated from materials and inks that are either coated or impervious to water, cleaning agents and any other environments to which the timer will become exposed in the medical environment. The advertising logo 502 is further protected or coated against abrasion and other forces to which it may be exposed in the hospital environment. In a preferred embodiment, the advertising logo 502 comprises a plurality of raised alphanumeric letters that are protected by raised edges or lips that minimize abrasive effects.

Referring to FIG. 5A, the timer button 400 represents an alternative embodiment, whose function is exactly as described in this document, but whose form factor is about that of a “dime-size”, or “quarter-size”, button, which can be attached via a clip or adhesive to be attached to a bandage, or to be held on a string or chain to be worn around an extremity or around the neck as a necklace. Furthermore, the button device could be used in the clinical laboratory as a timing device, and adhered to a blood vial or Petri dish using an adhesive backing 422.

FIG. 5B illustrates another embodiment of a timer button 500 wherein the timer button 500 can be clipped to a tablet, bed linen, clothing, or other thin structure. The timer button 500 comprises the substrate 402, the on-off switch 410, the LED 408, a clip 508 further comprising a plurality of jaws 506 and a plurality of compression tabs 504. The clip 508 can be opened manually by compressing the tabs 504 together to open the jaws 506.

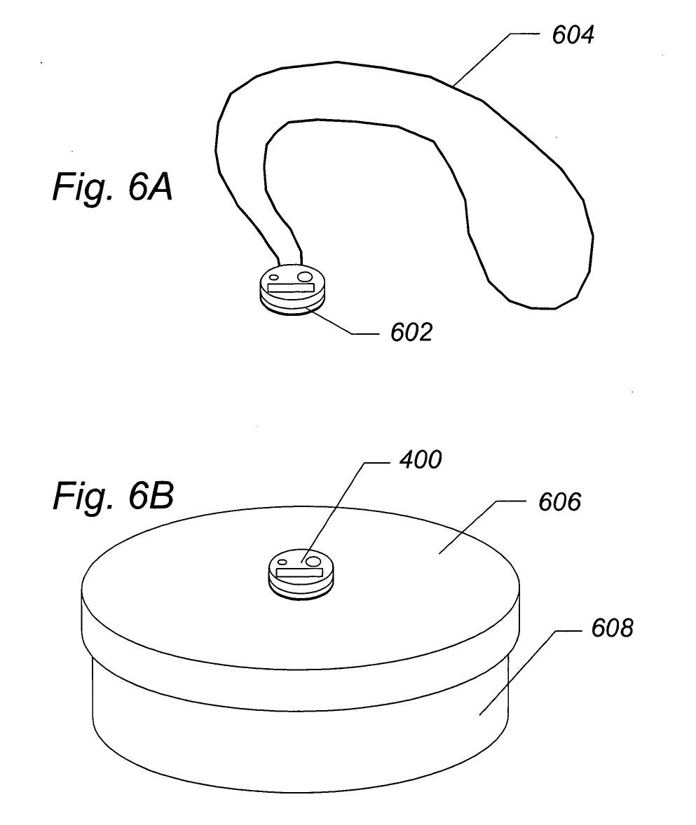

FIG. 6A illustrates another embodiment of the timer button 602 further comprising a lanyard, strap, cord, necklace, or other flexible linear loop 604 suitable for dangling around the neck of a patient or researcher.

FIG. 6B illustrates the timer button 400 of FIGS. 4A, 4B, and 5A affixed to the coverlid 606 of a Petri dish 608. The timer button 400 can be used to time down the growing cycle of cultures within the Petri dish in a convenient way. The label 502 can list the time to warning for the given timer button 400.

These active timing and warning devices will find critical application in a number of medical situations. Such situations include, but are not limited to, several examples as are listed in the following paragraphs. As listed below, the methods of use of these timing devices 100, 400, 500 can be important elements in patient care and improved accuracy of treatment and diagnosis.

The devices can be used as a tool to alert medical personnel of a clinically important time interval. Examples include a) removing and reinserting an intravenous line, b) changing of a wound dressing, c) removing and reinserting arterial and central venous catheter lines, inspection of wounds, d) adding insulin to an ambulatory pump or bedside pump, e) adding medication to an ambulatory or bedside pump, etc.

The devices disclosed herein can be used to measure body-surface temperature, and to alert medical personnel of a rise in temperature above baseline for a period of time that reduces false alarms. This would, thereby, alert medical personnel of a fever arising in a patient before the fever would otherwise be recognized. Furthermore, when placed in close proximity to a wound, the devices could alert medical personnel of an early infection (cellulitis or wound infection). It is the product of time and temperature, or the area under the curve (integral) of the time-temperature product, which can greatly reduce the number of false positive alerts.

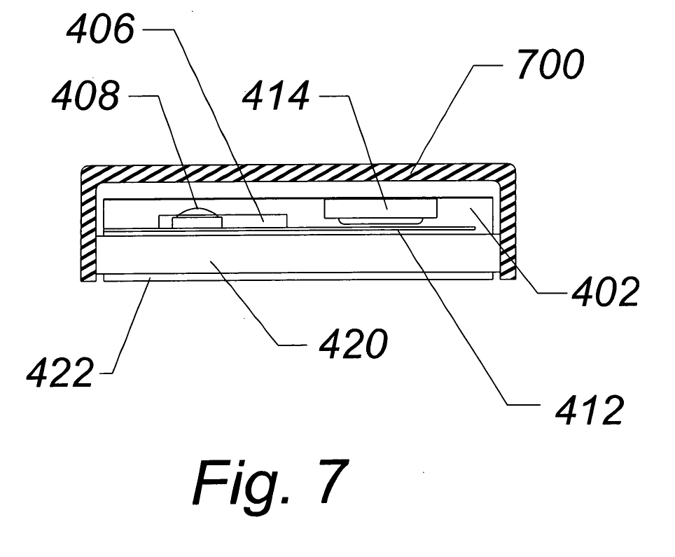

FIG. 7 illustrates a plastic snap-on, lid or closure device 700. This lid device 700 can snap on to the circuit board 412 and provide a seal for the device 400. In other embodiments, the snap-on lid or closure can comprise a conductor to operably engage the power supply with an electrical contact. The electrical contact can be separated from the battery or power supply with a sheet of insulator, paper or the like, for the purposes of shipping, storage, etc.

The timing devices 100, 400, 500, 602 disclosed herein can be used to establish a clinical protocol that differs from other protocols. For example, one surgeon may want a dressing changed in 24-hours, while another may desire a 48-hour period before undressing and inspecting. The medial/surgical ward nurse, heretofore, would have to remember each surgeon's preference, or check the medical chart to establish the appropriate interval. With certain embodiments of the disclosed devices, activated in the operating room upon completion of the procedure, the nurse will be reminded when the proper time arrives for changing the dressing. The period to wait before initiation of range of motion and exercise following joint surgery often varies amongst orthopedic surgeons. The ward nurse must refer to the medical record, or remember each surgeon's preference. Use of the disclosed devices, which visually flash or make audio warnings at the correct time, improves the process, thereby minimizing potential error.

There are many clinical time-dependent laboratory procedures. The technician, by adhering one of the timing devices 400 to the appropriate Petri dish or blood tube, will be reminded at the proper time to initiate or continue the procedure.

The timing bandage 100 can be used as a novel band-aid to attract attention of children, the device, in its simplest form, can be used as a promotional item to be given to children by Pediatricians, emergency rooms, clinics, doctors' offices, school nurses, etc. The band-aid 100 can be attached to an orthopedic cast or splinting device, wound wrap or bandage, etc. It can be set to flash continuously or upon movement of the body part to which it is attached. Such motion sensing flashing can be provided when the controller comprises a piezoresistive or piezoelectric switching element to initiate flashing. Other motion sensing means include level north sensors, accelerometers, and the like, all of which are capable of determining when motion occurs and initiating a visual or audio output sequence.

In the pediatric practice area, the band-aid 100 can be used to cover sutures, with the instruction that when the light begins to flash, the sutures should be removed. In addition, to appease an infant or adolescent who has received a vaccination via injection, a flashing band-aid could be used to cover the wound. The flashing bandage could be attractive to the patient and could serve to distinguish the patient among their peers.

In the dermatologic practice area, patients could be reminded to apply a cream to the area where this timing device 100 or 400 is applied. Often patients forget to apply a cream three times per day. The use of this timing device would be an easy reminder.

In the ophthalmologic practice area, this timing device 400, 500, or 602 could remind patients to apply eye drops at the appropriate time.

It is important to note that determination of core body temperature using measurements of skin temperature can be prone to error. Hence, certain embodiments of this device avoid this pitfall by sensing a change in temperature at the skin over a period of time. The microcontroller, differential amplifier, and associated circuitry is able to be programmed to detect a change in measured temperature that rises or falls greater than a predetermined acceptable deviation, and maintains or exceeds that deviation for a period of time. A fall in skin temperature greater than a deviation, or delta, can be used to indicate peripheral vasoconstriction, as seen in a hypotensive crisis, sepsis, hemorrhage, vaso-vagal reaction, etc. A rise in temperature can suggest infection, inflammation, anesthesia-induced hyperthermia, etc. Upon application to the skin the microcontroller will allow a predetermined period of “stabilization” of the thermistors, thermocouple, or other temperature sensing or measurement device. This time delay allows the electronic thermistor to thermally stabilize and also allows the skin vasculature to react to the application of the device and to stabilize. Following this period, the microcontroller then samples temperature either continuously or at predetermined intervals, either regular or random in nature. Should a change in temperature occur, and the time-temperature product reach a predetermined threshold, then the device will notify medical personnel with a visual display (flashing LED) or audible warning (beeper).

The visual display or audible beeper can be programmed to provide information concerning the quantitative rise or fall in temperature. For example, a single flash of the LED each 5seconds might indicate a 1-degree rise or fall from baseline. Two flashes each 5seconds would indicate a 2-degree change from baseline, and so on. In a similar way, the number of beeps could indicate the deviation of temperature from baseline. The important information that is conveyed to medical personnel is that there has been a change in temperature for a period of time, and that further investigation is indicated. The device is completely portable, and does not require connection to another electronic device for interpretation or display. However, this device may also be tethered or operably connected, by mans such as, but not limited to, radio frequency, infrared, microwave, ultraviolet radiation, or wire, to a central monitoring unit.

In a similar fashion, timing of critical clinical procedures can be accomplished using this self-contained device. The display of timing information can be accomplished using light emitting diodes, and does not require connection to another electronic device. The LEDs provide information relating to elapse time from start of timing, and a measure of time that has passed since the alert first occurred. For example, if a 48-hour timer is applied, the LEDs will first light to indicate operational integrity, and then enter sleep mode. At completion of the 48-hour period, the device will begin to flash. The LEDs can flash once each 5 seconds for the first hour, twice each 5 seconds for the second hour, 3 times each 5 seconds for the 3^{rd} hour, etc. In this way the clinician can determine how long it has been since the device first awoke. Other LED sequences could be programmed, depending on the desire of the end-user. Thus, information is contained in the sequence of alerts, thereby providing a simple and low-cost display method.

The device can be designed to monitor the patient's condition. The device can be tied to remote monitoring equipment using wi-fi, radio, blue-tooth, infrared, microwave, wired connections, or other methods of operable electronic or wireless communications.

In one embodiment this device may be packaged as a roll of tape. The individual units would be separated by a serration, thereby allowing easy access by tearing off of the individual unit. In another form the device could be individually packaged in a fashion similar to the common band-aid.

The device is designed to be used either once or multiple times. Information pertaining to time and temperature are immediately available to the medical practitioner through visual (LED) or auditory (beeper) alerts, or by vibration.

The applied voltage and current in the circuit described is direct current. There is no alternating current or voltage used other than seen in the crystal oscillator for timing purposes or for other specialized processing needs.

In another embodiment, the device used for timing purposes may take the shape of a coin. This circular-shaped device with adhesive backing could be applied to the patient on top of an existing dressing. In addition, the device could be adhered to the bedrail, bed headboard, intravenous (IV) bag, bladder catheter bag, etc. In this embodiment the coin-sized timing device alerts the clinical practitioner when a change in dressing, IV tubing, bladder catheter, etc., is required. In this embodiment the device could be packaged in a roll, with individual units “torn” off at pre-measured serrations.

Application of the coin-shaped device to laboratory equipment, such as a Petri dish, blood tube, culture bottle, etc., can provide notification to laboratory personnel that it is time to initiate a process.

Application of either the coin-shaped device or band-aid form to a ambulatory insulin pump would remind the patient that it is time to add insulin or to adjust the dose. It is well understood that young children and adolescents wearing ambulatory insulin pumps often forget to add insulin to the reservoir. Application of these timing devices to the site of the pump would provide additional assurance that insulin was added at the proper time.

The present invention may be embodied in other specific forms without departing from its spirit or essential characteristics. For example, the timer may or may not include a logo or advertisement and the number and duration of the countdown intervals may vary. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. An apparatus adapted to measure a pre-defined period and to alert medical personnel upon the conclusion of such period comprising:
a timer;
a visual display; wherein the visual display comprises one or more light emitting diodes; and
an adhesive backing system;
wherein the timer generates a timing sequence comprising a countdown, count-up, or elapsed-time protocol, and visually displays information addressing the duration to be timed and the amount of time elapsed.

2. The apparatus of claim 1 wherein said timer is temporarily affixed to the patient's bandage, skin, limb, neck, or other anatomic location.

3. The apparatus of claim 1 wherein said timer further comprises an advertising logo or message.

4. The apparatus of claim 1 wherein said timer comprises one or more mechanisms to initiate the timing sequence automatically upon removal of an adhesive cover strip, or upon activating a switch.

5. The apparatus of claim 1 wherein said timer further comprises an audible or visual or tactile signal to indicate that the timing sequence has been completed.

6. The apparatus of claim 1 wherein said timer further comprises a visual output device to indicate that the countdown has processed to zero.

7. The apparatus of claim 6 wherein said audible output is different depending on the time selected for the timing sequence.

8. The apparatus of claim 7 wherein said visual output is different depending on the time selected for the timing sequence.

9. A method of treating a wound comprising the steps of:
Applying a timing circuit to a bandage;
Removing an adhesive cover strip from the bandage to expose an adhesive surface;
Removing an electrically insulating barrier between two electrical contacts to initiate energy flow from a power source into a controller comprised by the timing circuit;
Adhering the bandage to a patient;
Performing a timing sequence within the timing circuit of the bandage, wherein the timing sequence comprises a countdown, count-up, or elapsed time program; and
Initiating a sequence of visual displays upon completion of the timing sequence.

10. The method of claim 9 wherein the step of initiating the sequence of visual displays comprises flashing an LED on and off.

11. The method of claim 9 further comprising the step of initiating a sequence of visual displays on more than one LED.

12. The method of claim 9 further comprising the step of pulsing the LED output such that the time since the timing sequence ended can be approximated by viewing the frequency sequence of the pulsing LED.

13. The method of claim 9 further comprising the step of initiating an audio output device upon completion of the timing sequence.

14. The method of claim 9 further comprising the step of measuring the temperature in the vicinity of the bandage.

15. The method of claim 9 further comprising the step of measuring the temperature of a region of the bandage and initiating a warning visual or audio sequence if the temperature deviates up or down from a baseline by an amount determined at the time of application of the device to the skin.

16. The method of claim 9 further comprising the step of affixing the insulating barrier to the adhesive cover strip so that when the adhesive cover strip is removed from the bandage, the timing circuit is activated.

17. The method of claim 9 wherein the step of adhering the bandage to the patient is replaced by clipping a timing circuit with all the same functions to a piece of cloth or other thin material.

18. The method of claim 9 wherein the bandage is replaced by a button-sized timer that can be worn around the neck of the patient.

19. The method of claim 9 wherein the bandage is replaced by a button-sized timer that can be adhered to the container of an experimental test setup.

20. An apparatus adapted to measure a pre-defined time period and to alert medical personnel upon the conclusion of such time period comprising:
a means for affixing the apparatus to a patient or object in the patient's room;
a means for providing electrical power to the apparatus;
a means for generating a timing sequence within the apparatus;
a means for alerting medical personnel when the timing sequence has reached a specified time interval; and
a means for initiating the timing sequence;
wherein the means for alerting the medical personnel comprises a sequence of light emitting diode activations that further comprise quantitative information regarding the length of the pre-defined time period and the time elapsed from the conclusion of the pre-defined time period to a present time.
